(19) United States
(12) Patent Application Publication
(10) Pub. No.: US 2025/0281512 A1
(43) Pub. Date: Sep. 11, 2025

(54) BUFFERING AGENT-CONTAINING COMPOSITIONS AND METHODS OF USING SAME

(71) Applicant: KNOW BIO, LLC, Morrisville, NC (US)

(72) Inventors: Drew Steven Folk, Cary, NC (US); Nathan Allan Stasko, Morrisville, NC (US); Rebecca Anthouard McDonald, Morrisville, NC (US); John Kelly Simons, Morrisville, NC (US); William Leroy Roberts, II, Morrisville, NC (US)

(73) Assignee: KNOW BIO, LLC, Morrisville, NC (US)

(21) Appl. No.: 18/856,841

(22) PCT Filed: Apr. 25, 2023
(86) PCT No.: PCT/US2023/019807

§ 371 Date: Oct. 14, 2024

Related U.S. Application Data
(60) Provisional application No. 63/335,822, filed on Apr. 28, 2022.

(51) Int. Cl. A61K 31/655; A61K 9/00; A61K 45/06; A61K 47/02; A61P 31/04

(52) U.S. Cl. CPC A61K 31/655; A61K 9/0078; A61K 45/06; A61K 47/02; A61P 31/04

(57) ABSTRACT

Provided herein is a composition including a buffering agent (e.g., a phosphate buffering agent) and an aqueous carrier, wherein the pH of the composition is maintained in a range of from 5.5 to 8.5, with a buffering strength of from 0.1 to 2.0 molar equivalents, and the osmolality of the composition is from 270 mOsm/kg to 1300 mOsm/kg. The buffering agent can further include an active pharmaceutical ingredient, such as a mucolytic agent, an antibiotic, an antiviral, a corticosteroid, a monoclonal antibody (mAb), an antifungal, or a nitric oxide releasing compound. Further described herein is a method for treating a respiratory disease in a subject, the method comprising administering to the subject an effective amount of a composition as described herein.

### CROSS REFERENCE TO PRIORITY APPLICATION

This application claims priority to U.S. Provisional Application No. 63/335,822, filed Apr. 28, 2022, which is incorporated herein by reference in its entirety.

### FIELD

The present disclosure relates to formulations including buffering agents, such as phosphate buffering agents, and a pH and osmolality controlled aqueous carrier, along with methods of their use in treating lung conditions in a subject.

### BACKGROUND

Nitric oxide has many therapeutic uses, including use as an antimicrobial agent. Nitric oxide gas can be administered to subjects via inhalation; however, such administration is difficult, time-consuming, and potentially toxic to the individual based on the required amount and duration of exposure necessary. Nitric-oxide releasing compounds have also been explored as therapeutic agents for delivering nitric oxide. Such compounds must be formulated, however, to ensure adequate stability prior to and during administration by the subject and subsequently adequate levels of nitric oxide delivered to the lung at a desirable release rate.

### SUMMARY

Provided herein is composition comprising a buffering agent, such as a phosphate buffering agent, and an aqueous carrier. The aqueous carrier is pH and osmolality controlled. Specifically, the pH of the composition is maintained in a range of from 5.5 to 8.5, with a buffering strength of from 0.1 to 2.0 molar equivalents, and the osmolality of the composition is from 270 mOsm/kg to 1300 mOsm/kg. Optionally, the buffering agent is a phosphate buffering agent comprising potassium phosphate. The composition can be administered by inhalation, orally, intravenously, or any other suitable route of administration.

The composition can further comprise an active pharmaceutical ingredient, such as a water-soluble active pharmaceutical ingredient. Optionally, the active pharmaceutical agent comprises a mucolytic agent, an antibiotic, an antiviral, a corticosteroid, a monoclonal antibody (mAb), or an antifungal. Optionally, the active pharmaceutical ingredient comprises a nitric oxide (NO) releasing compound. In some cases, the NO-releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation. Optionally, the compound has the following structure:

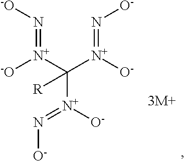

wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H; a, b, c, d, e, and f are each independently selected from an integer of 0, 1, 2, 3, 4, 5, 6, 7, 8, 9, or 10; and M+ is a pharmaceutically-acceptable cation, wherein a ratio of the compound to the cation is such that the overall net charge of the compound is neutral.

In some examples, the cation is selected from the group consisting of sodium, potassium, lithium, calcium, magnesium, ammonium, and substituted ammonium. Optionally, the compound has the following structure:

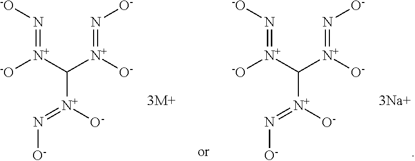

In some cases, a molar equivalents concentration ratio of the phosphate buffering agent to the compound in the composition is at least 0.4:1. Optionally, the phosphate buffering agent maintains the pH of the composition in a range of from 5.5 to 8.0 (e.g., from 6.0 to 8.0, from 6.5 to 8.5, from 6.5 to 8.0, from 6.7 to 7.5, or from 7.0 to 7.5). Optionally, the composition has an osmolality of 270 mOsm/kg to 1300 mOsm/kg (e.g., from 270 mOsm/kg to 900 mOsm/kg, from 300 mOsm/kg to 800 mOsm/kg, or from greater than 300 mOsm/kg to 750 mOsm/kg).

As described above, the phosphate buffering agent can be a potassium phosphate buffer. In some examples, the phosphate buffering agent is substantially free from sodium phosphate. In some cases, the composition is substantially free from carbonate buffers, hydrochloric acid, sulphuric acid, or citric acid.

The compositions described herein can further comprise one or more additives. The one or more additives can optionally comprise one or more preservatives, salts, chelators, viscosity modifiers, stabilizers, surfactants, antioxidants, or cosolvents.

Optionally, the molar equivalents concentration ratio of the phosphate buffering agent to the compound in the composition is at least 0.1:1 (e.g., at least 0.2:1, at least 0.3:1, at least 0.4:1, at least 0.5:1, at least 0.6:1, or from 0.65:1 to 2.5:1). The compound can be present in an amount of from 0.1 mg/mL to 200 mg/mL (e.g., from 1 mg/mL to 100 mg/mL or from 10 mg/mL to 50 mg/mL). In some examples, the compound in the composition has a total releasable nitric oxide (NO) storage in a range of 0.1-23.0 μmol of NO per mg of the compound. Optionally, the compound has a NO release half-life in the range of 0.01-24 hours. In some cases, the compound has a total duration of NO release in a range of 0.1-60 hours. The compound can optionally have a total NO release of 0.1-8.0 μmol of NO per mg of the compound after 4 hours of release initiation.

Also provided herein is a stable composition of a diazeniumdiolate compound (NONOate) in aqueous conditions, comprising a composition comprising a phosphate buffering agent, an aqueous carrier, and a nitric oxide releasing compound comprising at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation, wherein the pH of the composition is maintained in a range of from 5.5 to 8.5 and the osmolality of the composition is from 270 mOsm/kg to 1300 mOsm/kg.

Further described herein is a method for treating a respiratory disease in a subject, comprising administering to the subject an effective amount of a composition as described herein. Optionally, the composition is an inhalable composition that is administered by inhalation. Optionally, the inhalable composition is administered using a nebulizer, a metered dose inhaler, or a dry powder inhaler. Optionally, the respiratory disease is an acute or chronic lung infection caused by a microbial pathogen from one or more of bacteria, viruses, or fungi. The subject can optionally have asthma, chronic obstructive pulmonary disease, emphysema, acute bronchitis, cystic fibrosis, pneumonia, bronchiectasis, or bronchiolitis. In other examples, the composition is administered orally, intravenously, or by any other suitable route of administration.

The details of one or more embodiments are forth in the drawings and the description below. Other features, objects, and advantages will be apparent from the description and drawings, and from the claims.

### DESCRIPTION OF THE DRAWINGS

FIG. 1 is a graph showing compound (MD3) consumption as a function of formulation pH over time. The horizontal line at 90% represents the lower limit for the MD3 concentration at the end of delivery. The vertical line at 30 minutes represents the upper practical time limit for nebulization delivery to occur.

FIG. 2 is a graph showing compound efficacy (MD3 efficacy) in HEPES buffer, as demonstrated by in vitro minimal inhibitory concentration (MIC) and minimal bactericidal concentration (MBC), against Pseudomonas aeruginosa strain K (PAK) as a function of pH.

FIG. 3 is a graph showing compound (MD3) formulation pH stability as a function of phosphate equivalents.

FIG. 4 is a graph showing the impact of hypothetical compound (MD3) formulations on solution osmolality at a fixed concentration of buffering agent.

FIG. 5 is a graph showing hypothetical compound (MD3) formulations to achieve the desired doses in animal studies. In each group of data points, the uppermost point represents 8 h, the middle point represents 6 h, and the lowest point represents 4 h.

FIG. 6 is a graph showing the impact of pH (6.0 or 7.0) on NO flux over an eight-hour period. In the figure, the upper line represents pH 6.0 and the lower line represents pH 7.0.

FIGS. 7A-C contain graphs showing the impact of pH (pH 6.5, FIG. 7A; pH 7.5, FIG. 7B; pH 8.5, FIG. 7C) on NO flux over an eight-hour period for compound (MD3) in 50 mM HEPES buffer solution.

FIG. 8A is a graph demonstrating the relationship between buffer concentration and osmolality for a HEPES buffer (lower line) and a phosphate buffer (upper line). FIGS. 8B and 8C contain graphs showing the impact of buffer (phosphate buffer, FIG. 8B; HEPES, FIG. 8C) on NO flux over an eight-hour period for a MD3 containing composition.

FIGS. 9A-C contain graphs showing the time to kill Pseudomonas aeruginosa strain K (PAK) using varying concentrations of compound (MD3) in HEPES buffer, including 0.125 mg/mL (FIG. 9A), 0.0625 mg/mL (FIG. 9B), and 0.03125 mg/mL (FIG. 9C).

FIGS. 10A-C contain graphs showing the time to kill Pseudomonas aeruginosa strain K (PAK) using varying concentrations of compound (MD3) in phosphate buffer, including 0.125 mg/mL (FIG. 10A), 0.0625 mg/mL (FIG. 10B), and 0.03125 mg/mL (FIG. 10C).

FIG. 11 is a graph showing the nitric oxide (NO) release needed to kill PAK was measured using varying concentrations of compound (MD3) at pH values of 6.5, 7.5, and 8.5.

### DETAILED DESCRIPTION

Described herein are compositions including a buffering agent (such as a phosphate buffering agent) and a carrier, along with methods of their use in treating lung conditions in a subject. The compositions, including the buffering agent and carrier, possess therapeutic effects and are useful in treating one or more lung conditions. The therapeutic effects of the compositions described herein can be modified by including one or more active pharmaceutical agents in the composition. Each of the active pharmaceutical agents can be selected such that the therapeutic impact of the agent combines with the buffering agent and carrier to provide an additive or synergistic effect in treating lung conditions, thus creating a tunable formulation.

For example, the diazeniumdiolate-containing compounds as described herein are useful for the treatment of respiratory diseases and lung infections caused by bacteria, viruses, and fungi. Upon dissolution in aqueous conditions, the diazeniumdiolate-containing compounds release nitric oxide (NO) with release rates dependent on solution pH, which then exerts a variety of biological effects in a concentration dependent manner. The compounds have broad spectrum antimicrobial activity and/or can modulate host inflammation. When the compounds described herein are formulated as an aqueous solution for nebulized delivery, the solution characteristics and their impact on the drug substance must be considered to obtain a safe, effective, and stable drug product prior to and during administration to the subject. As described herein, those attributes include pH, tonicity, viscosity, buffer strength, as well as the impact of any additives such as preservatives, chelators, stabilizers, and surfactants, antioxidants, or cosolvents. The compositions can be formulated for administration via inhalation, orally, intravenously, or any other suitable route of administration. The tunable compositions are further described below.

### 1. Compositions

A composition for use in the methods described herein includes a buffering agent, such as a phosphate buffering agent, and an aqueous carrier. As further described herein, the pH of the composition is maintained in a range of from 5.5 to 8.5, with a buffering strength of from 0.1 to 2.0 molar equivalents, and the osmolality of the composition is from 270 mOsm/kg to 1300 mOsm/kg. The formulations described herein can be tailored to a desired pH, while controlling the osmolality, buffer equivalency and viscosity required for successful nebulization using desired devices (e.g., vibrating mesh).

Surprisingly, the compositions described herein, including the phosphate buffering agent and aqueous carrier, can enhance orally or intravenously delivered antibiotic uptake into lung tissue and can modulate disease on its own (i.e., without any further pharmaceutical agents). For example, the compositions described herein can be used for airway clearance in muco-obstructive airway diseases by thinning mucous and hydrating lung tissue. The compositions can also include salt concentrations that can reduce or eliminate biofilms. Further, the cation of the buffering agent may impact proton pumps of airway epithelial cells to modulate epithelial lining fluid pH. The compositions can further include one or more active pharmaceutical agents and other additives, as further described below. The composition in combination with one or more active pharmaceutical agents result in additive or synergistic effects with respect to antimicrobial activity.

### Buffering Agent

Buffering agents can be included to control the pH of the composition. In some examples, the buffering agent is included to maintain the pH of the composition from 5.5 to 8.5. For example, the buffering agent can be included to maintain the pH of the composition from 5.5 to 8.0, from 6.0 to 8.0, from 6.5 to 8.5, from 6.5 to 8.0, from 6.7 to 7.5, or 7.0 to 7.5 (e.g., 7.4).

The buffering agent can have a buffering strength of from 0.1 to 2.0 molar equivalents (e.g., from 0.1 to 1.5 molar equivalents, from 0.2 to 1.25 molar equivalents, or from 0.3 to 1.0 molar equivalents). For example, the buffering agent can have a buffering strength of 0.1 molar equivalents, 0.2 molar equivalents, 0.3 molar equivalents, 0.4 molar equivalents, 0.5 molar equivalents, 0.6 molar equivalents, 0.7 molar equivalents, 0.8 molar equivalents, 0.9 molar equivalents, 1.0 molar equivalents, 1.1 molar equivalents, 1.2 molar equivalents, 1.3 molar equivalents, 1.4 molar equivalents, 1.5 molar equivalents, 1.6 molar equivalents, 1.7 molar equivalents, 1.8 molar equivalents, 1.9 molar equivalents, or 2.0 molar equivalents.

In general, the buffering agent can be any buffering agent generally regarded as safe for use as inactive ingredients suitable for administration (e.g., via inhalation). In some embodiments, the buffering agent for use in the compositions described herein includes a phosphate buffering agent. Examples of suitable phosphate buffering agents include, for example, 0.01-1 M phosphate buffering agents. Optionally, the phosphate buffering agent is a potassium phosphate buffer. The counter cation of the buffering agent for use in the compositions can be selected to enhance the biologic activity of the composition or to minimize complexity in the analytical characterization of the composition.

Generally, sodium is an acceptable counter cation, but in certain examples, the use of sodium should be limited or excluded. Specifically, certain examples of the compounds described herein, such as MD3 (as further described below), includes sodium cations as counterions. When measuring the amount of the compound in a certain formulation, such as an aerosol formulation, the amount of the sodium cation is calculated. Under these circumstances, the presence of sodium in a buffering agent (e.g., a sodium phosphate buffering agent) would obfuscate the compound calculation. In such examples, the composition is free from or substantially free from sodium phosphate. In other examples, the presence of potassium in a buffering agent may have an impact on proton pumps and the resulting pH of the epithelial lining fluid when administered to a human. Potassium does not increase the alkalinity of the composition, which is beneficial as an increase in alkalinity would interfere with NO release from a nitric oxide releasing compound.

One or more buffering agents can be included in the composition, including acetate buffers, benzoate buffers, citrate buffers, lactate buffers, maleate buffers, and tartrate buffers. Optionally, the one or more buffering agents includes a HEPES ((4-(2-hydroxyethyl)-1-piperazineethanesulfonic acid) buffering agent.

In some examples, the composition is substantially free from carbonate buffering agents. In some examples, the composition is substantially free from hydrochloric acid, sulphuric acid, or citric acid. As used herein, the term “substantially free” from an indicated component (e.g., carbonate buffers, hydrochloric acid, sulphuric acid, and/or citric acid), means that the pharmaceutical composition can include less than 1%, less than 0.1%, less than 0.01%, less than 0.001%, or less than 0.0001% of the component (e.g., carbonate buffering agents, hydrochloric acid, sulphuric acid, and/or citric acid) based on the weight of the pharmaceutical composition. In other examples, the composition can include sodium phosphate, carbonate buffering agents, hydrochloric acid, sulphuric acid, and/or citric acid in amounts suitable to control the pH of the composition, as described above.

### Carriers

The composition described herein also includes a carrier. As used herein, the term carrier encompasses any excipient, diluent, filler, salt, buffer, stabilizer, solubilizer, lipid, stabilizer, or other material well known in the art for use in pharmaceutical formulations. Suitable liquid carriers can be aqueous carriers. Aqueous carriers include water, ethanol, glycerol, alcoholic/aqueous solutions, emulsions, or suspensions. Water or an aqueous carrier is preferred.

The carrier can optionally be included in an amount of 95 wt. % to 99 wt. %. For example, the amount of carrier present in the composition can be 95 wt. %, 95.5 wt. %, 96 wt. %, 96.5 wt. %, 97 wt. %, 97.5 wt. %, 98 wt. %, 98.5 wt. %, or 99 wt. %.

Optionally, the amount of carrier in the composition is controlled such that the composition, prepared from the carrier, the buffering agent, any active pharmaceutical ingredients, and/or additives, has an osmolality of 270 mOsm/kg to 1300 mOsm/kg (e.g., from 300 mOsm/kg to 900 mOsm/kg). For example, the composition can have an osmolality of 270 mOsm/kg, 280 mOsm/kg, 290 mOsm/kg, 300 mOsm/kg, 310 mOsm/kg, 320 mOsm/kg, 330 mOsm/kg, 340 mOsm/kg, 350 mOsm/kg, 360 mOsm/kg, 370 mOsm/kg, 380 mOsm/kg, 390 mOsm/kg, 400 mOsm/kg, 410 mOsm/kg, 420 mOsm/kg, 430 mOsm/kg, 440 mOsm/kg, 450 mOsm/kg, 460 mOsm/kg, 470 mOsm/kg, 480 mOsm/kg, 490 mOsm/kg, 500 mOsm/kg, 550 mOsm/kg, 600 mOsm/kg, 650 mOsm/kg, 700 mOsm/kg, 750 mOsm/kg, 800 mOsm/kg, 850 mOsm/kg, 900 mOsm/kg, 950 mOsm/kg, 1000 mOsm/kg, 1050 mOsm/kg, 1100 mOsm/kg, 1150 mOsm/kg, 1200 mOsm/kg, 1250 mOsm/kg, or 1300 mOsm/kg. The compositions having the controlled osmolality as described herein allow for respiratory diseases to be effectively treated, such as through the thinning of mucous in a subject.

### Active Pharmaceutical Ingredient(s)

In some cases, the compositions described herein can further include one or more active pharmaceutical ingredients. Such compositions can also be used to treat respiratory diseases, such as an acute or chronic lung infection caused by a microbial pathogen from one or more of bacteria, viruses, or fungi. The active pharmaceutical ingredient can be a water-soluble active pharmaceutical ingredient. Optionally, the active pharmaceutical ingredient is nitric oxide and can be included in the composition in the form of a compound that releases nitric oxide (NO) (e.g., nitric oxide donors, nitric oxide releasing prodrugs, or compounds necessary to facilitate the endogenous generation of nitric oxide).

In some cases, the compositions can include at least one nitric oxide releasing compound. Certain compositions include a nitric oxide releasing compound having at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

As stated above, the compounds described herein can include at least one nitric oxide releasing functional group. Although various NO donors (e.g., diazeniumdiolates, S-nitrosothiols, metal nitrosyls, organic nitrates) are known to provide for controlled exogenous NO release, the diazeniumdiolate functional group (NONOate) in the compounds disclosed herein are attractive because of their good stability and facile storage, and because they spontaneously undergo proton-triggered dissociation under physiological conditions to regenerate nitric oxide. Certain compounds include two diazeniumdiolate groups on one carbon atom, each having a charge and each with an associated pharmaceutically-acceptable cation to balance the charge on the diazeniumdiolate groups. The compounds are small molecules (having a molecular weight of 500 g/mol or less, without the cation, as further described below) that release nitric oxide (NO) and exhibit antimicrobial characteristics.

Optionally, the compound has the following structure, as represented by Formula I:

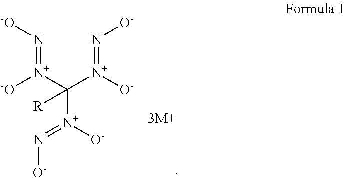

In Formula I, R is hydrogen, deuterium, C₁₋₁₂ alkyl, aryl, heteroaryl, alkylaryl, arylalkyl, or carbonyl. Optionally R is substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H, wherein a, b, c, d, e, and f are each independently selected from an integer of 0, 1, 2, 3, 4, 5, 6, 7, 8, 9, or 10.

Additionally in Formula I, M+ is a cation. For example, M+ can be a pharmaceutically acceptable cation. Optionally, the cation is selected from the group consisting of sodium, potassium, lithium, calcium, magnesium, and quaternary ammonium salts (e.g., ammonium or substituted ammonium).

In these compositions, a ratio of the compound to the cation is such that the overall net charge of the compound is neutral. In cases where M+ is a cation with a valence other than one, a ratio of the compound to the cation is such that the total positive charge equals the total negative charge. By way of example, for a compound having a total charge of negative three, and a cation with a total charge of positive one, there would be one compound and three cations.

For example, the compound can be represented by Structure I-A, as shown below:

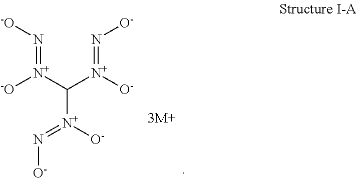

As shown above in Structure I-A, the compound has a total charge of negative three. Therefore, three cations (i.e., 3 M+) are present to balance the charge of the compound (i.e., the total positive charge equals the total negative charge).

An example of Structure I-A includes the following compound:

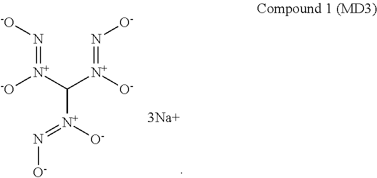

The compound can have a molecular weight below 500 g/mol, not including the associated cation (e.g., the associated pharmaceutically-acceptable cation). For example, the compound can have a molecular weight of 450 g/mol or less, 400 g/mol or less, 350 g/mol or less, 300 g/mol or less, 250 g/mol or less, or 200 g/mol or less. Optionally, the molecular weight of the compound, excluding the associated cation, can be from 100 g/mol to below 500 g/mol, from 120 g/mol to 450 g/mol, from 150 g/mol to 400 g/mol, or from 175 g/mol to 350 g/mol.

Additional details regarding the mechanism of action of the compounds described herein, including their nitric oxide delivery properties, and advantageous properties of the compounds (e.g., storage stability) are described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bonded through a single, terminal ether linkage. The term aryloxy as used herein is an aryl group bonded through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bonded through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

The C-diazeniumdiolates described herein are pH-triggered NO-releasing donors (also referred to herein as NO-releasing compounds or NO-releasing agents). Reacting with protons under physiological conditions (e.g., 37° C., pH 7.4), 1 mole of Compound 1 (MD3) generates two moles of NO and 2 to 3 moles of nitroxyl compounds.

In several embodiments, the NO-releasing compounds are stable at a variety of temperatures from frozen to room temperature 25° C. (e.g., −20° C., 0° C., 5° C., 20° C., etc.) and are stable for prolonged storage periods (e.g., 10 hours, 20 hours, 22 hours, 25 hours, 30 hours, etc., days such as 1 day, 3 days, 5 days, 6 days, 7 days, 15 days, 30 days, 45 days, etc., weeks such as 1 week, 2 weeks, 3 weeks, 4 weeks, 6 weeks, 8 weeks, etc., months such as 1 month, 2 months, 3 months, 4 months, 5 months, 6 months, etc., or even years (1 year, 2 years, or greater)).

In some cases, the compound has a total releasable NO storage in a range of 0.1 μmol-23.0 μmol of NO per mg of the compound (e.g., from 0.1 μmol to 15 μmol per mg of the compound, from 0.5 μmol to 7.5 μmol per mg of the compound, from 1 μmol to 7.0 μmol per mg of the compound, from 1.5 μmol to 6.5 μmol per mg of the compound, from 2.0 μmol to 6.0 μmol per mg of the compound, from 2.5 μmol to 5.5 μmol per mg of the compound, or from 3.0 μmol to 5.0 μmol per mg of the compound). For example, the total releasable NO storage of the compounds for use in the composition can be 0.1 μmol, 0.2 μmol, 0.3 μmol, 0.4 μmol, 0.5 μmol, 0.6 μmol, 0.7 μmol, 0.8 μmol, 0.9 μmol, 1.0 μmol, 1.1 μmol, 1.2 μmol, 1.3 μmol, 1.4 μmol, 1.5 μmol, 1.6 μmol, 1.7 μmol, 1.8 μmol, 1.9 μmol, 2.0 μmol, 2.1 μmol, 2.2 μmol, 2.3 μmol, 2.4 μmol, 2.5 μmol, 2.6 μmol, 2.7 μmol, 2.8 μmol, 2.9 μmol, 3.0 μmol, 3.1 μmol, 3.2 μmol, 3.3 μmol, 3.4 μmol, 3.5 μmol, 3.6 μmol, 3.7 μmol, 3.8 μmol, 3.9 μmol, 4.0 μmol, 4.1 μmol, 4.2 μmol, 4.3 μmol, 4.4 μmol, 4.5 μmol, 4.6 μmol, 4.7 μmol, 4.8 μmol, 4.9 μmol, 5.0 μmol, 5.1 μmol, 5.2 μmol, 5.3 μmol, 5.4 μmol, 5.5 μmol, 5.6 μmol, 5.7 μmol, 5.8 μmol, 5.9 μmol, 6.0 μmol, 6.1 μmol, 6.2 μmol, 6.3 μmol, 6.4 μmol, 6.5 μmol, 6.6 μmol, 6.7 μmol, 6.8 μmol, 6.9 μmol, 7.0 μmol, 7.1 μmol, 7.2 μmol, 7.3 μmol, 7.4 μmol, 7.5 μmol, 7.6 μmol, 7.7 μmol, 7.8 μmol, 7.9 μmol, 8.0 μmol, 8.1 μmol, 8.2 μmol, 8.3 μmol, 8.4 μmol, 8.5 μmol, 8.6 μmol, 8.7 μmol, 8.8 μmol, 8.9 μmol, 9.0 μmol, 9.1 μmol, 9.2 μmol, 9.3 μmol, 9.4 μmol, 9.5 μmol, 9.6 μmol, 9.7 μmol, 9.8 μmol, 9.9 μmol, 10.0 μmol, 10.1 μmol, 10.2 μmol, 10.3 μmol, 10.4 μmol, 10.5 μmol, 10.6 μmol, 10.7 μmol, 10.8 μmol, 10.9 μmol, 11.0 μmol, 11.1 μmol, 11.2 μmol, 11.3 μmol, 11.4 μmol, 11.5 μmol, 11.6 μmol, 11.7 μmol, 11.8 μmol, 11.9 μmol, 12.0 μmol, 12.1 μmol, 12.2 μmol, 12.3 μmol, 12.4 μmol, 12.5 μmol, 12.6 μmol, 12.7 μmol, 12.8 μmol, 12.9 μmol, 13.0 μmol, 13.1 μmol, 13.2 μmol, 13.3 μmol, 13.4 μmol, 13.5 μmol, 13.6 μmol, 13.7 μmol, 13.8 μmol, 13.9 μmol, 14.0 μmol, 14.1 μmol, 14.2 μmol, 14.3 μmol, 14.4 μmol, 14.5 μmol, 14.6 μmol, 14.7 μmol, 14.8 μmol, 14.9 μmol, 15.0 μmol, 15.1 μmol, 15.2 μmol, 15.3 μmol, 15.4 μmol, 15.5 μmol, 15.6 μmol, 15.7 μmol, 15.8 μmol, 15.9 μmol, 16.0 μmol, 16.1 μmol, 16.2 μmol, 16.3 μmol, 16.4 μmol, 16.5 μmol, 16.6 μmol, 16.7 μmol, 16.8 μmol, 16.9 μmol, 17.0 μmol, 17.1 μmol, 17.2 μmol, 17.3 μmol, 17.4 μmol, 17.5 μmol, 17.6 μmol, 17.7 μmol, 17.8 μmol, 17.9 μmol, 18.0 μmol, 18.1 μmol, 18.2 μmol, 18.3 μmol, 18.4 μmol, 18.5 μmol, 18.6 μmol, 18.7 μmol, 18.8 μmol, 18.9 μmol, 19.0 μmol, 19.1 μmol, 19.2 μmol, 19.3 μmol, 19.4 μmol, 19.5 μmol, 19.6 μmol, 19.7 μmol, 19.8 μmol, 19.9 μmol, 20.0 μmol, 20.1 μmol, 20.2 μmol, 20.3 μmol, 20.4 μmol, 20.5 μmol, 20.6 μmol, 20.7 μmol, 20.8 μmol, 20.9 μmol, 21.0 μmol, 21.1 μmol, 21.2 μmol, 21.3 μmol, 21.4 μmol, 21.5 μmol, 21.6 μmol, 21.7 μmol, 21.8 μmol, 21.9 μmol, 22.0 μmol, 22.1 μmol, 22.2 μmol, 22.3 μmol, 22.4 μmol, 22.5 μmol, 22.6 μmol, 22.7 μmol, 22.8 μmol, 22.9 μmol, or 23.0 μmol per mg of the compound.

The compound can have a total duration of NO release, upon activation, in a range of 0.1-60 hours. In some cases, the NO release may occur over a period of about 0.1 hours, 0.25 hours, 0.5 hours, 1 hour, 2 hours, 3 hours, 4 hours, 5 hours, 10 hours, 15 hours, 20 hours, 24 hours, 36 hours, 48 hours, or 60 hours. In some embodiments, within 2 hours of being added to a PBS buffer solution, the compounds release greater than or equal to about: 25%, 50%, 75%, 85%, 90%, 95%, 100%, or ranges including and/or spanning the aforementioned values, their total wt. % of bound NO. Optionally, the compound has a total NO release of 0.1-8.0 μmol of NO per mg of the compound after 4 hours of the initiation of NO release (also referred to as “activation”).

In some embodiments, the compounds have a release rate per hour using chemiluminescent based nitric oxide detection of less than or equal to about: 0.2%, 0.5%, 1.0%, 1.5%, 2.5%, 5.0%, 10%, or ranges including and/or spanning the aforementioned values.

Optionally, the compound for use in the compositions described herein has a NO release half-life in the range of 0.01-24 hours. In several embodiments, the NO release half-life is equal to or at least about: 0.01 hours, 0.1 hours, 0.25 hours, 0.5 hours, 1 hour, 2 hours, 3 hours, 4 hours, 5 hours or ranges including and/or spanning the aforementioned values. In some embodiments, the NO release occurs in less than or equal to about: 0.01 hours, 0.1 hours, 0.25 hours, 0.5 hours, 1 hour, 2 hours, 3 hours, 4 hours, 5 hours, 10 hours, 15 hours, 20 hours, 24 hours or ranges including and/or spanning the aforementioned values.

In some examples of compositions as described herein, the compound is present in an amount of from 0.1 mg/mL to 200 mg/mL (e.g., from 1 mg/mL to 100 mg/mL, from 1 mg/mL to 90 mg/mL, from 1 mg/mL to 80 mg/mL, from 1 mg/mL to 70 mg/mL, from 1 mg/mL to 60 mg/mL, from 5 mg/mL to 55 mg/mL, from 10 mg/mL to 50 mg/mL, from 15 mg/mL to 45 mg/mL, or from 20 mg/mL to 40 mg/mL). For example, the concentration of the compound in the composition can be 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 35 mg/mL, 40 mg/mL, 45 mg/mL, 50 mg/mL, 55 mg/mL, 60 mg/mL, 65 mg/mL, 70 mg/mL, 75 mg/mL, 80 mg/mL, 85 mg/mL, 90 mg/mL, 95 mg/mL, 100 mg/mL, 105 mg/mL, 110 mg/mL, 115 mg/mL, 120 mg/mL, 125 mg/mL, 130 mg/mL, 135 mg/mL, 140 mg/mL, 145 mg/mL, 150 mg/mL, 155 mg/mL, 160 mg/mL, 165 mg/mL, 170 mg/mL, 175 mg/mL, 180 mg/mL, 185 mg/mL, 190 mg/mL, 195 mg/mL, or 200 mg/mL.

Optionally, such as for diazeniumdiolate-containing compounds, the amount of buffering agent (e.g., phosphate buffering agent) added to the composition is such that the molar equivalents concentration ratio of the buffering agent to the compound in the composition is at least 0.1:1 (e.g., at least 0.2:1, at least 0.3:1, at least 0.4:1, at least 0.45:1, at least 0.5:1, or at least 0.6:1). For example, the molar equivalents concentration ratio of the buffering agent to the compound in the composition can be from 0.65:1 to 2.5:1. In some cases, the molar equivalents concentration ratio of the buffering agent to the compound in the composition can be 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, or 2.5:1.

Further examples of suitable active pharmaceutical agents may include, for example, mucolytic agents, antibiotics, antivirals, antifungals, corticosteroids, and/or monoclonal antibodies (mAbs) as described below. Other agents that produce an additive or synergistic therapeutic effect with the active pharmaceutical agent can also be included in the formulation.

### Optional Additives

The compositions described herein can further include one or more additives. The one or more additives can include, for example, one or more preservatives, salts, chelators, stabilizers, surfactants, antioxidants (e.g., N-acetylcysteine or glutathione), and/or cosolvents. Optionally, the preservatives for use in the compositions can include thymol and/or benzalkonium chlorides, such as alkyl dimethyl benzyl ammonium chlorides, alkyl dimethyl (phenylmethyl) quaternary ammonium chlorides, ammonium alkyl dimethyl (phenylmethyl) chlorides, or ammonium alkyl dimethyl benzyl chlorides. The composition, if desired, can also contain wetting or emulsifying agents, lubricants, glidants, emollients, humectants, thickeners, and/or flavoring agents.

In some cases, the one or more additives can include viscosity-reducing agents, natural and synthetic anti-biofilm agents (e.g., chitosan), biofilm dispersing agents, natural and synthetic anti-quorum-sensing agents (e.g., autoinducer-2 or N-acyl homo-serine lactones), siderophores, iron chelators, iron mimetics (e.g., gallium (Ga) and gallium-containing compounds, such as gallium azoles (Ga-azoles)), anti-persister cell agents (e.g., 4-(4,7-di-methyl-1,2,3,4-tetrahydro-naphthalene-1-yl) pentanoic acid (DMNP)), antimicrobial peptides (AMPs) (e.g., LL-37 or lactoferricin), efflux pump inhibitors, and/or bacteriophage therapy.

Optionally, the additives can be present in an amount of less than 1 wt. %. For example, the amount of the additive can be less than 0.9 wt. %, less than 0.8 wt. %, less than 0.7 wt. %, less than 0.6 wt. %, less than 0.5 wt. %, less than 0.4 wt. %, less than 0.3 wt. %, less than 0.2 wt. %, or less than 0.1 wt. %. The amount of additive can optionally be 0.1 to 0.9 wt. %, 0.2 to 0.8 wt. %, or 0.3 to 0.7 wt. %.

Viscosity modifiers can optionally be included in the compositions as described herein. Optionally, the viscosity modifiers can be included in the compositions in an amount of up to 5 wt. % (e.g., 0.1 wt. % to 5 wt. %, 0.5 wt. % to 4.5 wt. %, 1.0 wt. % to 4.0 wt. %, 1.5 wt. % to 3.5 wt. %, or 2.0 wt. % to 3.0 wt. %). For example, the viscosity modifier can be 0.1 wt. %, 0.5 wt. %, 1.0 wt. %, 1.5 wt. %, 2.0 wt. %, 2.5 wt. %, 3.0 wt. %, 3.5 wt. %, 4.0 wt. %, or 4.5 wt. %, and 5.0 wt. %.

### II. Pharmaceutical Compositions

In some cases, the composition is a pharmaceutical composition. In cases where the composition is a pharmaceutical composition, the compositions can include one or more of the compounds described herein and a pharmaceutically acceptable carrier. Furthermore, the one or more compounds described herein can be combined with other agents, including treatments for lung, digestive, hepatic, and biliary tract related diseases and disorders. For example, in the case of cystic fibrosis, the compositions described herein can be combined with mucus thinning drugs (e.g., domase alfa, N-acetyl cysteine, and hypertonic saline), bronchodilators (e.g., metaproterenol sulfate, pirbuterol acetate, salmeterol, albuterol, and terbutaline sulfate), P2Y2-receptor agonists (e.g., denufosol), and agents that target nonsense mutations (e.g., PTC124). Further examples of additional agents that can be combined with the compounds described herein include antibiotics (e.g., aminoglycosides, antipseudomonal penicillins, and cephalosporins), antimicrobial drugs (e.g., rifabutin), ethambutol, clarithromycin, clofazimine, aztreonam, steroidal and nonsteroidal anti-inflammatory drugs (e.g., ibuprofen and prednisone), pentoxifylline, dornase alfa, or ursodeoxycholic acid.

The one or more compounds described herein, with or without additional agents, can be provided in the form of an inhaler or nebulizer for inhalation therapy. In some cases, the one or more compounds described herein, with or without additional agents, can be administered using a metered dose inhaler or a dry powder inhaler. As used herein, inhalation therapy refers to the delivery of a therapeutic agent, such as the compounds described herein, in an aerosol form to the respiratory tract (i.e., pulmonary delivery). As used herein, the term aerosol refers to very fine liquid or solid particles suspended in a gas (e.g., air) and delivered to a site of therapeutic application. When a pharmaceutical aerosol is employed, the aerosol contains the one or more compounds described herein, which can be dissolved, suspended, or emulsified in a mixture of a fluid carrier and/or a propellant, in the case of a metered dose inhaler. The aerosol can be in the form of a solution, suspension, emulsion, powder, or semi-solid preparation. Aerosols employed are intended for administration as fine, solid particles or as liquid mists via the respiratory tract of a patient.

As described above, the one or more compositions described herein can be provided with a nebulizer, which is an instrument that generates very fine liquid particles of substantially uniform size in a gas. The liquid containing the one or more compounds described herein can be dispersed as droplets about 5 mm or less in diameter (e.g., 5 μm or less or 1 μm to 5 μm in diameter) in the form of a mist. The small droplets can be carried by a current of air or oxygen through an outlet tube of the nebulizer. The resulting mist can penetrate into the respiratory tract of the patient.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

The preparation of pharmaceutically acceptable carriers and formulations containing these materials is described in, e.g., Remington: The Science and Practice of Pharmacy, Adeboye Adejare ed., 23rd Ed., Academic Press (2021). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, gels, pastes, creams, and lotions. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

As noted above, the compositions can include one or more of the compounds described herein or pharmaceutically acceptable salts thereof. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.01 to about 200 mg/kg of body weight of active compound per day, which may be administered in a single dose or in the form of individual divided doses, such as from 1 to 4 times per day. Alternatively, the dosage amount can be from about 0.05 to about 190 mg/kg of body weight of active compound per day, about 0.1 to about 180 mg/kg of body weight of active compound per day, about 0.25 to about 175 mg/kg of body weight of active compound per day, about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day. Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

### III. Methods of Use

Provided herein are methods of administering the compositions described herein to a subject. As used herein, the terms “administering” and “administration” refer to any method of providing composition to a subject. Such methods are well known to those skilled in the art and include, but are not limited to, administration by inhalation, oral administration, transdermal administration, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. The compositions described herein can be administered therapeutically; that is, administered to treat an existing disease or condition. In some examples, the composition can be administered prophylactically; that is, administered for prevention of a disease or condition.

In some examples, provided herein are methods of treating a respiratory disease in a subject. The methods include administering to the subject an effective amount of a composition as described herein. The respiratory disease can include an acute or chronic lung infection caused by a microbial pathogen from one or more of bacteria, viruses, or fungi. Effective amount, when used to describe an amount of compound in a method, refers to the amount of a compound that achieves the desired pharmacological effect or other biological effect. The effective amount of the composition can be, for example, delivery of enough compound to the target region of the lung to achieve the minimal inhibitory concentration (MIC) or the minimal bactericidal concentration (MBC) against a particular bacterial strain in the epithelial lining fluid (ELF).

As demonstrated in the examples herein, bacterial killing is concentration- and pH-dependent, but may not be dependent on the total NO released. For example, a composition as described herein at a pH of 8.0 or lower (e.g., 7.5, 7.0, or 6.5) may effectively kill bacteria at a certain concentration, whereas more than twice the concentration may not kill bacteria at a pH greater than 8.0 (e.g., 8.5).

In some examples, the respiratory diseases are caused by bacteria in humans, e.g., pediatric and geriatric populations, and in animals, e.g., veterinary applications. In some cases, the bacteria include at least one of Gram-positive bacteria, Gram-negative bacteria, and atypical bacteria.

Optionally, the bacteria is a Gram-positive bacteria species, such as an Actinomyces species, a Bacillus species, a Clostridium species, a Corynebacterium species, an Enterococcus species, a Leuconostoc species, a Micrococcus species, a Nocardia species, a Propionibacterium species, a Staphylococcus species, or a Streptococcus species.

Optionally, the bacteria is a Gram-negative bacteria species, such as an Acinetobacter species, an Aeromonas species, an Alcaligenes/Achromobacter species, a Bacteroides species, a Bartonella species, a Bordetella species, a Borrelia species, a Brevundimonas species, a Brucella species, a Burkholderia species, a Campylobacter species, a Citrobacter species, a Coxiella species, an Ehrlichia species, an Enterobacter species, an Escherichia species, a Francisella species, a Haemophilus species, a Helicobacter species, a Klebsiella species, a Leclercia species, a Legionella species, a Leptospira species, a Listeria species, a Moraxella species, a Morganella species, a Neisseria species, an Orientia species, a Pantoea species, a Paracoccus species, a Prevotella species, a Proteus species, a Providencia species, a Pseudomonas species (e.g., Pseudomonas aeruginosa), a Ralstonia species, a Rickettsia species, a Roseomonas species, Salmonella species, a Serratia species, a Shigella species, a Sphingomonas species, a Stenotrophomonas species, a Treponema species, a Ureaplasma species, a Vibrio species, or a Yersinia species.

Optionally, the bacteria is an atypical bacteria species, such as a Mycobacteria species, a Chlamydia/Chlamidophila species, or a Mycoplasma species. In some cases, the bacteria can include antibiotic-resistant bacteria, such as antibiotic-resistant Burkholderia cepacia, carbapenem-resistant Enterobacteriaceae (CRE) gut bacteria, drug-resistant Campylobacter, drug-resistant non-typhoidal Salmonella, drug-resistant Shigella, multi-drug-resistant Acinetobacter, multi-drug-resistant Escherichia coli, multi-drug-resistant Klebsiella pneumoniae, multi-drug-resistant Neisseria gonorrhoeae, multidrug-resistant Pseudomonas aeruginosa, antibiotic-resistant Clostridium difficile, drug-resistant Streptococcus pneumoniae, clindamycin-resistant Group B Streptococcus, erythromycin-resistant Group A Streptococcus, methicillin-resistant Staphylococcus aureus (MRSA), vancomycin-resistant Staphylococcus aureus (VRSA), and vancomycin-resistant Enterococcus (VRE).

In some examples, the respiratory diseases are caused by viruses in humans, e.g., pediatric and geriatric populations, and in animals, e.g., veterinary applications. The virus can be an RNA or a DNA virus. Optionally, the virus can be an enveloped or non-enveloped virus. Examples of enveloped viruses include, but are not limited to, human immunodeficiency virus (HIV), vesicular stomatitis virus (VSV), herpes simplex viruses (HSV-1 and HSV-2), and other herpes viruses, for example, varicella-zoster virus (VZV), EBV, equine herpes virus (EHV), influenza virus and human cytomegalovirus (HCMV). Examples of non-enveloped viruses include, but are not limited to, papilloma virus (PV) and adenoviruses (AV).

In some examples, the respiratory disease is caused by a respiratory viruses including those that cause upper respiratory tract infections and lower respiratory tract infections. The viruses can include, for example, coronaviruses, rhinoviruses, Respiratory Syncytial Virus, paramyxoviruses, such as parainfluenza viruses, for example HPIV-1, HPIV-2, HPIV-3, HPIV-4, HPIV-4a and HPIV-4b, and other influenza viruses, such as influenza A and influenza B. Diseases resulting from infections by these viruses, such as common colds, influenza, bronchiolitis, pneumonia, croup, bronchitis, pharyngitis, laryngitis, otitis media and sudden acute respiratory system (SARS), can also be treated according to some of the present methods.

Optionally, the respiratory diseases are caused by fungi in humans, e.g., pediatric and geriatric populations, and in animals, e.g., veterinary applications. Representative fungal infections that can be treated include Candida albicans, drug resistant Candida albicans, Candida glabrata, Candida krusei, Candida guilliermondii, Candida auris, Candida tropicalis, Aspergillus niger, Aspergillus terreus, Aspergillus fumigatus, and/or Aspergillus flavus.

Further examples of microbial pathogens, including bacteria, viruses, or fungi, that can be the source of a respiratory disease for treatment according to the methods described herein are described in PCT/US2021/016841, entitled “Nitric Oxide-Releasing Antibacterial Compounds, Formulations, and Methods Pertaining Thereto;” PCT/US2021/016854, entitled “Nitric Oxide-Releasing Antibacterial Compounds, Formulations, and Methods Pertaining Thereto;” and/or PCT/US2021/016869, entitled “Nitric Oxide-Releasing Antibacterial Compounds, Formulations, and Methods Pertaining Thereto;” each of which are incorporated herein by reference in their entireties.

The methods of treating a respiratory disease can further include selecting a subject having a respiratory disease or at risk of developing a respiratory disease. Optionally, the subject has asthma, chronic obstructive pulmonary disease, emphysema, acute bronchitis, cystic fibrosis, pneumonia, bronchiectasis, or bronchiolitis. The methods of treatment described herein can further include treatment with one or more additional agents (e.g., an antibiotic, an antiviral, and/or an antifungal). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

Suitable antibiotics can include any antibiotic effective for treating a respiratory disease including, for example, tetracyclines (e.g., minocycline), quinolones (e.g., ciprofloxacin, levofloxacin, and nalidixic acid), aminoglycosides (e.g., amikacin, gentamycin, kanamycin, and tobramycin), carbapenems (e.g., meropenem), cephalosporins (e.g., ceftriaxone and ceftazidime), macrolides (e.g., erythromycin and clarithromycin), polypeptides (e.g., colistin and polymxin B), sulfonamides (e.g., sulfamethoxazole), glycylcyclines (e.g., tigecycline), beta lactams (e.g., penams), lipopeptides (e.g., daptomycin), oxazolidinones (e.g., linezolid), and trimethoprim.

For example, the compounds or compositions or pharmaceutically acceptable salts thereof as described herein can be combined into a pharmaceutical composition with an additional antibiotic, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; aztreonam; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocycline hydrochloride; cetophenicol; chloramphenicol; chloramphenicol palmitate; chloramphenicol pantothenate complex; chloramphenicol sodium succinate; chlorhexidine phosphanilate; chloroxylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium; pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

Suitable antivirals include, for example, abacavir, acyclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, tenofovir, tenofovir disoproxil, tipranavir, trifluridine, trizivir, tromantadine, truvada, valaciclovir, valganciclovir, vicriviroc, vidarabine, viramidine zalcitabine, zanamivir, and/or zidovudine.

Suitable antifungals agents include, for example, amphotericin B, fluconazole, flucytosine, itraconazole, ketoconazole, clotrimazole, econozole, griseofulvin, miconazole, nystatin, and/or ciclopirox.

Optionally, the respiratory disease can be caused by a respiratory infectious viruses (e.g., infectious diseases due to respiratory infectious viruses such as influenza virus, rhino virus, corona virus, parainfluenza virus, RS virus, adeno virus, reovirus and the like), herpes zoster caused by herpes virus, diarrhea caused by rotavirus, viral hepatitis, AIDS and the like. The bacterial infectious disease is not particularly limited and includes, for example, infectious diseases caused by Bacillus cereus, Vibrio parahaemolyticus, Enterohemorrhagic Escherichia coli, Staphylococcus aureus (e.g., methicillin-resistant Staphylococcus aureus), Salmonella, Botulinus, Candida and the like.

In some examples, the respiratory disease can be an inflammatory lung disease or a chronic lung disease having a dysregulated inflammatory process that can be modulated by, for example, nitric oxide. For example, the respiratory disease can be asthma, COPD, chronic bronchitis, bronchiectasis, or cystic fibrosis. In some examples, the respiratory disease can be a lung disease having a cardiovascular component that can be modulated by, for example, nitric oxide. For example, the respiratory disease can be atherosclerosis, post-angioplasty, restenosis, coronary artery diseases or angina. Optionally, the subject can have nontuberculous mycobacterial (NTM) lung disease and/or Lady Windermere syndrome (LWS). In some cases, the subject can be a lung transplant patient.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of a respiratory disease), during early onset (e.g., upon initial signs and symptoms of a respiratory disease), or after an established respiratory disease. Prophylactic administration can occur for several hours to years prior to the manifestation of symptoms of an infection. Prophylactic administration can be used, for example, in the preventative treatment of subjects or surfaces exposed to Pseudomonas aeruginosa or to prevent exacerbations. Therapeutic treatment involves contacting the subject with a therapeutically effective amount of the compositions as described herein.

### IV. Stable Compositions and Kits

Also provided herein is a stable composition including a composition as described herein. In some cases, the composition is lyophilized. Optionally, the stable composition described herein includes a diazeniumdiolate compound in aqueous conditions. In some cases, the composition can also include a bulking agent. In some examples, the stable composition can include a composition comprising a buffering agent (e.g., a phosphate buffering agent), an aqueous carrier, and a nitric oxide releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation, wherein the pH of the composition is maintained in a range of from 5.5 to 8.5 and the osmolality of the composition is from 270 mOsm/kg to 1300 mOsm/kg.

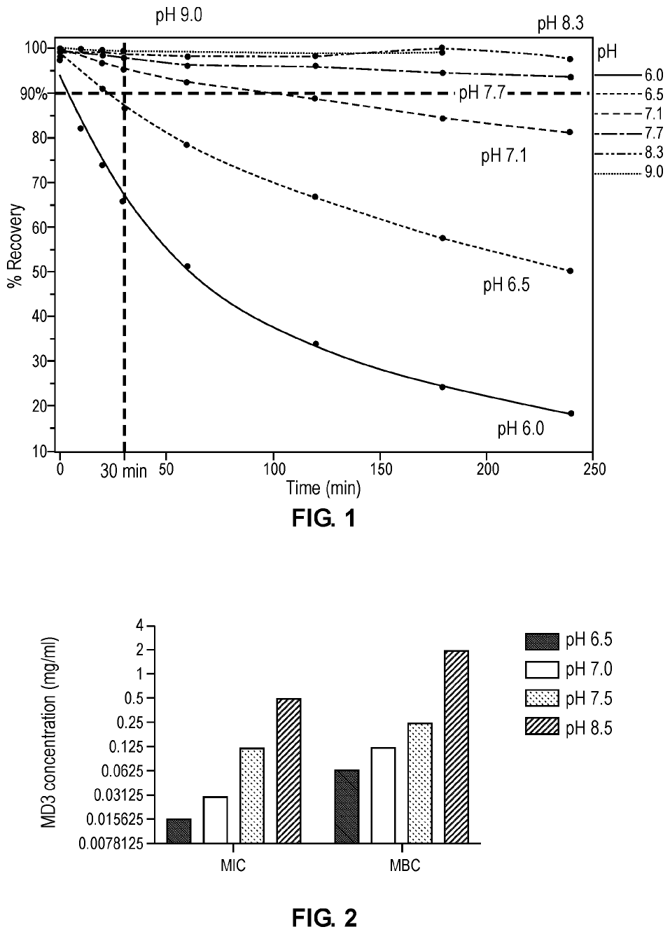

When in the form of an aqueous composition, as described herein, the compositions described herein are appropriately stable to be suitable for effective administration to a subject. By way of example, for diazeniumdiolate compounds, at least 90% of the diazeniumdiolate compound (e.g., MD3) remains intact (i.e., at least 90% of the NO within the diazeniumdiolate compound has not been released) for a suitable period of time. In some examples, the suitable period of time is at least 20 minutes, at least 30 minutes, at least 40 minutes, at least 50 minutes, or at least 1 hour. Optionally, the suitable period of time for diazeniumdiolate compound stability in an aqueous medium is from 20 minutes to 10 hours, from 25 minutes to 5 hours, or from 30 minutes to 4.5 hours. Exemplary parameters for a stable composition as described herein are outlined, for example, in FIG. 1. Optionally, the composition is an inhalable composition.

The stable compositions can optionally be provided in the form of a kit. A kit can include any of the compositions described herein. For example, a kit can include a compound of Formula I and a carrier (e.g., a pharmaceutically acceptable carrier).

A kit can include a means for delivery. In some cases, a kit can include a means for delivery by inhalation (e.g., an inhaler or a nebulizer). A kit can additionally include directions for use of the kit (e.g., instructions for treating a subject or contacting a surface), one or more containers (for the compound(s), composition(s), or second biofilm inhibiting agent(s), a means for administering the compounds or compositions, and/or a carrier.

Optionally, the stable composition kit can include one or more containers. A first container can include the buffering agent (e.g., a phosphate buffering agent) and a carrier. Optionally, the kit can include a second container including one or more active pharmaceutical ingredients. In some cases, the final formulation is the content of the first container (i.e., the buffering agent and carrier). In some cases, the final formulation is the combination of the first and second containers (i.e., the buffering agent, carrier, and one or more active pharmaceutical ingredients). In some examples, the contents of the first and second containers are mixed a period of time prior to administration (e.g., prior to administration by inhalation). The period of storage time for the prepared formulation can vary based on the storage temperature and formulation particulars. By way of example, the storage period can be for example 1 hour or less, 45 minutes or less, 30 minutes or less, 20 minutes or less, or 15 minutes or less when the prepared formulation is stored at a temperature ranging from 5° C. to 20° C.

As used herein the terms treatment, treat, or treating refer to a method of reducing one or more symptoms of a disease or condition. Thus in the disclosed method, treatment can refer to a 5%, 10%, 20%, 30%, 40%, 50%, 60%, 70%, 80%, 90%, or 100% reduction in the severity of one or more symptoms of the disease or condition. For example, a method for treating a disease is considered to be a treatment if there is a 5% reduction in one or more symptoms or signs of the disease in a subject as compared to a control. As used herein, control refers to the untreated condition. Thus the reduction can be a 5%, 10%, 20%, 30%, 40%, 50%, 60%, 70%, 80%, 90%, 100%, or any percent reduction in between 5% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 5%, 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the subject matter described herein which are apparent to one skilled in the art.

### Example 1: Formulation Design

Formulation components and their requisite amounts were designed to develop aqueous solutions of the compounds described herein. In particular, the formulations in the study were designed for nebulized delivery to treat bronchiectasis and other potential respiratory diseases. Compound MD3 (shown below; referred to herein as “MD3”)) was used as a model to design a suitable vehicle for compound delivery:

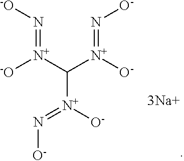

Upon activation at neutral pH and elevated temperatures, MD3 releases nitric oxide (NO), which exerts a variety of biological effects including having broad-spectrum antimicrobial activity. Because MD3 is being formulated as an aqueous solution for nebulized delivery, the solution characteristics and their impact on the drug substance must be considered to obtain a safe, effective, and stable drug product. Those attributes include pH, tonicity, viscosity, buffer strength, as well as the impact of any additives such as preservatives, chelators, stabilizers, and surfactants, antioxidants, or cosolvents.

### pH Design

One of the considered attributes of the MD3 solution for nebulization is the pH. At basic pH (>8.5), NO release from MD3 is negligible. However, the rate of NO release and subsequent antibacterial activity increases dramatically as the pH is lowered to neutral or acidic. Selecting the appropriate formulation pH to enable release of NO in the lung while maintaining stability until delivery is a subtle balance.

To maximize MD3 stability prior to dosing, the drug substance is manufactured as a basic solution with a pH˜11 and stored frozen to limit premature release of NO. The drug substance solution would not be amenable to inhalation delivery directly since dosing a solution at this pH can result in irritation of the respiratory epithelial layer. Prior to dosing, the pH of the formulation for dosing can be lowered to better match physiological conditions for safety and tolerability purposes. Therefore, prior to dosing, the MD3 solution is mixed with a vehicle to generate the activated formulation for nebulization.

Studies on MD3 stability as a function of formulation pH were performed. MD3 (10 mg/mL, 38 mM)) was incubated for 4 hours in various 200 mM phosphate buffers ranging from pH 6.0 to 8.3 at room temperature. At various time points, an aliquot of buffered solution was removed and diluted 100× in pH 9.0 ammonium bicarbonate solution to quench the reaction and the aliquot was analyzed for MD3 content via HPLC. The % recovery of MD3 was calculated at each timepoint based on the t=0 result. An MD3 sample maintained at pH 9.0 in ammonium bicarbonate buffer was used as a control. The results demonstrate that the recovery of drug in the formulation decreases over time in a pH dependent fashion. See FIG. 1. In FIG. 1, the horizontal line at 90% represents the lower limit for the MD3 concentration at the end of delivery, and the vertical line at 30 minutes represents the upper practical time limit for nebulization delivery to occur. If a formulation having greater than 90% intact MD3 at a relevant clinical dosing time of 30 minutes is desired, the formulation pH should not be less than 7.0 (FIG. 1). Formulations prepared at pH values of 6.0 and 6.5 are not considered viable formulations because the formulations release NO too quickly; as such, the formulations do not have acceptable in-use stability to deliver a consistent dose over a 30 min period. Formulations prepared at pH values of 8.3 and 9.0 are not considered viable formulations because the alkalinity may cause irritation or inflammation of lung tissues, and NO release at these pH values is negligible over 24 hours.

In vitro efficacy studies favor a lower formulation pH for enhanced antimicrobial properties. MD3 was formulated in 50 mM HEPES to a final pH of either 6.5, 7.0, 7.5, or 8.5. Susceptibility testing was performed following the Clinical and Laboratory Standards Institute (CLSI) standard methods. Briefly, P. aeruginosa strain K was streaked Tryptic Soy Agar plates and incubated at 37° C. overnight. Colonies were aseptically swabbed and resuspended in 1×PBS, then diluted to 5×105 CFU/ml in 2× cation-adjusted Mueller-Hinton Broth. Bacteria were added to 2-fold serial dilutions of MD3 formulations in a 96-well plate and incubated in a 37° C. incubator for 18-24 hours. After incubation, the minimum inhibitory concentration (MIC) values were determined as the lowest drug concentration that did not support bacterial growth (i.e., no turbidity). To determine the minimal bactericidal concentration (MBC), 100 μL of the remaining clear wells were plated onto TSA plates, and these were incubated at 37° C. overnight. A 3-log reduction in CFU/ml was considered the MBC.

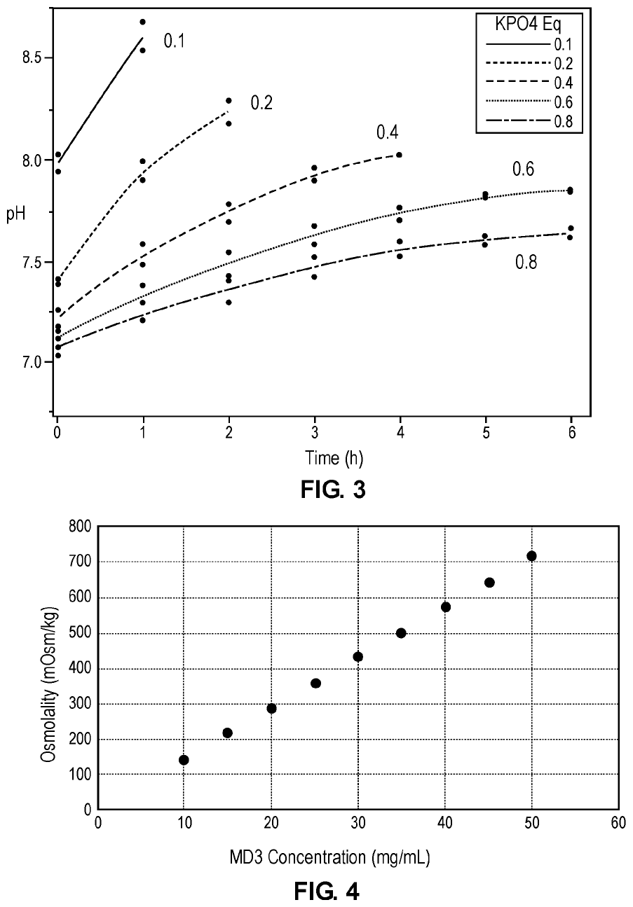

As shown in FIG. 2 and Table 1, compound (MD3) efficacy is pH-dependent. The in vitro minimal inhibitory concentration (MIC) and the minimal bactericidal concentration (MBC) of MD3 against Pseudomonas aeruginosa strain K (PAK) decreased by approximately 32-fold when the pH is reduced from 8.5 to 6.5. A lower pH results in less compound (MD3) needed to both inhibit the growth of PAK and eradicate PAK.

TABLE 1

| Condition | MIC (mg/mL) | MBC (mg/mL) | pH after 24 hours |
|---|---|---|---|
| pH 6.5, HEPES | 0.0156 | 0.0625 | ~6.7 |
| pH 7.0, HEPES | 0.03125 | 0.125 | ~7.0 |
| pH 7.5, HEPES | 0.125 | 0.25 | ~7.6 |
| pH 8.0, HEPES | 0.5 | 2 | ~8.6 |

The pH of healthy lungs is understood in the field to be near neutral. In patients with bronchiectasis and other inflammatory infections, exhaled breath condensate containing microdroplets of the extracellular lining fluid of the lung, suggest a more acidic environment, with a pH of 6.4-6.8. The acidification of MD3 once delivered can provide increased efficacy against bacterial infection. MD3 dosed via intratracheal (IT) administration in SCID mice showed no visible adverse effects at 100 mg/kg MD3 when the formulation pH was above 8.5. Conversely, a similar study where the formulation pH was maintained at 7.4, showed MD3 was lethal at 88 mg/kg. These in vivo studies show that the natural buffer capacity of the lung combined with clearance mechanisms from the mucosa may not allow for enough time for an alkaline dose of MD3 to be neutralized and release a therapeutic dose of NO prior to clearance or absorption. When considering a balance of stability with efficacy for example, a formulation pH of 7.0±0.3 can elicit the antimicrobial properties desired upon delivery with appropriate formulation stability to support a human dosing period.

### Buffer Identity and Strength

Complementing pH selection is the type and strength of the buffer system used to maintain the chosen pH. Currently approved inhalation solutions typically use three types of acids as pH modifiers: sulfuric acid, hydrochloric acid, and citric acid. Sulfuric acid and hydrochloric acid are strong acids useful for modifying the pH, but do not offer any buffering capacity. These are not ideal for an MD3 formulation for two reasons. First, the vehicle prior to mixing with MD3 would be exceedingly acidic (pH<2), and upon initial mixing with MD3 would induce excessive off-gassing of NO until the pH is neutralized. Second, the release of NO from MD3 is a proton-mediated process and therefore, in a neutral, unbuffered solutions, the pH will increase as NO is released until it reaches 8.5-9.0 where the NO release if effectively quenched and efficacy may be compromised. To maintain an acceptable pH from initial mixing of the formulation throughout dosing, a suitable buffer is necessary. While citric acid does offer some buffering capacity, with pKa's at 3, 5, and 6 it does not effectively buffer the pH above 7.0 as desired for delivery of the NO-releasing compounds described herein.

There are several options for buffers to be used with NO-releasing compounds with effective capacity at neutral pH, including organic buffers as shown in Table 2. MD3 formulations prepared with buffers with buffering ranges below 7 will generally lead to much more rapid NO release. For inhalation delivery, slower release is preferred to account for the timeframes required to deliver a dose (1 min to 30 min) and the desire to deliver NO over an extended period of time. NONOate compounds prepared with buffers with buffering ranges above 8 are unable to release enough NO to be effective as an antimicrobial agent.

TABLE 2

| | | | Suitable for |
|---|---|---|---|
| | | pH | use with |
| Buffer | Buffer Name | Range | NONOate |
| MES | 2-(N-morpholino)ethanesulfonic acid | 5.7-6.5 | No |
| BIS-TRIS | Bis-tris methane | 5.9-7.1 | No |
| ACES | N--2-aminoethanesulfonic acid | 6.1-7.3 | No |
| PIPES | piperazine-N,N′-bis(2-ethanesulfonic acid) | 6.2-7.2 | No |
| MOPSO | 2-Hydroxy-3-morpholinopropanesulfonic acid | 6.3-7.5 | Yes |
| BES | (N,N-bis(2-hydroxyethyl)-2-aminoethanesulfonic | 6.4-7.7 | Yes |
| | acid) | | |
| MOPS | (3-(N-morpholino)propanesulfonic acid) | 6.5-7.9 | Yes |
| TES | 2-{[1,3-Dihydroxy-2-(hydroxymethyl)propan-2- | 6.9-8.1 | Yes |
| | yl]amino}ethane-1-sulfonic acid | | |
| HEPES | (4-(2-hydroxyethyl)-1-piperazineethanesulfonic | 6.9-8.1 | Yes |
| | acid) | | |
| TRIS | tris(hydroxymethyl)aminomethane | 7.0-9.0 | Yes |
| (H)EPPS | 3-[4-(2-Hydroxyethyl)piperazin-1-yl]propane-1- | 7.3-8.6 | Yes |
| | sulfonic acid | | |
| TRICINE | N-tris(Hydroxymethyl)methylglycine | 7.7-8.8 | No |
| BICINE | N,N-Bis(2-hydroxyethyl)glycine | 7.8-9.0 | No |
| TAPS | ([tris(hydroxymethyl)methylamino]propanesulfonic | 7.8-9.1 | No |
| | acid) | | |
| GLYGLY | Glycylglycine | 7.9-9.5 | No |
| AMPSO | N-(1,1-Dimethyl-2-hydroxyethyl)-3-amino-2- | 8.3-9.6 | No |
| | hydroxypropanesulfonic acid | | |
| CHES | N-Cyclohexyl-2-aminoethanesulfonic acid | 8.6-10.0 | No |
| CAPS | N-cyclohexyl-3-aminopropanesulfonic acid | 9.7-11.1 | No |

As outlined in Table 2, several buffers, including MOPSO, BES, MOPS, TES, HEPES, TRIS, and (H)EPPS buffers, possess a suitable buffering range. However, the selected buffer for use in certain methods described herein, including NO delivery through an NO-releasing compound such as MD3, must also be physiologically compatible and effective.

The most physiologically-relevant buffers are carbonate and phosphate (not shown in Table 2). Carbonate buffer, while excellent for maintaining pH in blood plasma, is not as effective in maintaining pH under atmospheric conditions because the conjugate acid, carbonic acid, will decompose in water and release carbon dioxide from solution. Phosphate buffers offer excellent pH stability under atmospheric conditions and, depending on the molar ratio utilized, provides excellent buffering capacity in the desired control pH range of 6.5 to 8.5. Phosphate was selected as the buffer for maintaining pH 7.0 for MD3 solution for nebulization.

As discussed, while the pH of the formulation has a direct effect on the stability and likely efficacy of the formulation, the concentration of phosphate determines how long the pH is maintained in a desirable range while MD3 is releasing NO. Once the buffering capacity is exceeded, the pH will increase until NO release is quenched. A study was performed evaluating the pH stability of the MD3 formulation over time with varying molar equivalents of phosphate. MD3 concentrations of 46 mg/mL and 23 mg/mL (177 mM and 89 mM respectively) were tested while phosphate levels were varied from 0.1 to 0.8 equivalents of MD3 at pH 7.0.

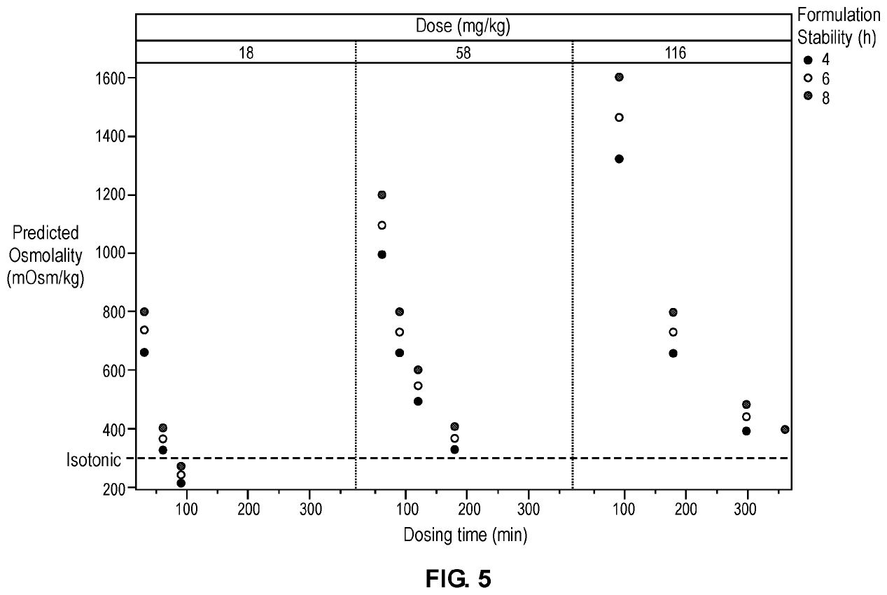

Briefly, MD3 at two concentrations (23.3 mg/mL and 46.5 mg/mL) was incubated in phosphate buffers at pH 7.0 at various concentrations ranging from 9-142 mM (representing 0.1-0.8 equivalents of PO4/mM MD3) for 4 hours or until the pH exceeded 8.0. At various time points, the pH was recorded for each solution to understand how the pH changes as function of buffer concentration. The results from the two MD3 concentrations were pooled due a similar response in both samples and are shown in FIG. 3.

As shown in FIG. 3, the starting pH and rate of pH change are directly related to the equivalents of phosphate in solution with higher levels of phosphate enabling a starting pH closer to 7.0 and increased pH stability throughout the testing interval. The results from both MD3 formulation strengths were pooled because there is not a significant difference between the 46 and 23 mg/mL formulations.

Formulations prepared at a pH of 7.0, but with no more than 0.2 equivalents of buffer capacity, are not considered viable formulations for inhalation because the pH is not adequately buffered and quickly rises. As a result, after a very short time the alkalinity of the solution will halt NO release. Formulations prepared at a pH of 7.0, but with 0.4 to 0.6 equivalents of buffer capacity, are viable formulations but are considered to be unoptimized regarding their ability to maintain pH control over extended periods.

Where a pH range of 7.0-7.5 is desirable, the amount of phosphate in the formulation can be at least 0.6 equivalents of the MD3 concentration to maintain acceptable formulation pH through the duration of dosing in an animal study (1-4 hours). For human studies, where the dosing time will be considerably shorter (<30 minutes), a phosphate concentration of 0.4 equivalents is considered.

### Osmolality

An important consideration of any drug product formulation for inhalation is the osmolality, which is the number of solute particles dissolved in a solvent. Ideally, the dosing solution would be isotonic with the physiological environment, including, for example, an osmolality around 300 mOsm/kg. Both hyper- and hypotonic solutions are known to induce bronchoconstriction, coughing, and irritation of the lung mucosa.

Interestingly, inhalation of hypertonic saline at various concentrations has been approved by the FDA to help clear mucous from the lungs of CF patients, so hypertonic formulations can be beneficial in this space if administered for a short duration. Therefore, an MD3 formulation that is isotonic or slightly hypertonic (>300 mOsm/kg) would likely be acceptable.

The osmolality of the MD3 drug product for human clinical studies are determined by the concentration of MD3, phosphate buffer, any formulation additives or stabilizers that are dissolved in the nebulization solution. To simply the formulation and better understand the effects of MD3 directly in non-GLP animal studies, only MD3 and phosphate were included in the formulation.

A model was developed based on empirical evidence to help predict the osmolality of a designed formulation of compound (in this example, MD3) and phosphate. The resulting formula is described below:

Formulation Osmolality (mOsm/kg)=3.3*Compound (mM)+2.2*Phosphate (mM)

Formulations prepared for non-GLP toxicity and efficacy studies were evaluated for osmolality against the model. The osmolality was measured via freezing point depression using an osmometer. The close agreement between the predicted osmolality and observed is shown in Table 3.

TABLE 3

| | | | Predicted | Measured |
|---|---|---|---|---|
| Study | | Component | Osmolality | Osmolality |
| No. | Formulation | Summary | (mOsm/kg) | (mOsm/kg) |
| 1 | 45:55 Mixture of | 160 mM MD3 | 836 | 884 |
| | MD3 and 255 mM | 140 mM Phosphate | | |
| | Potassium Phosphate | (0.88 eq) | | |
| | Buffer, pH 7.0 | | | |
| 2 | 70:30 Mixture of | 247 mM MD3 | 1211 | 1228 |
| | MD3 and 597 mM | 179 mM Phosphate | | |
| | Potassium Phosphate | (0.72 eq) | | |
| | Buffer, pH 7.0 | | | |
| | (MTD) | | | |
| 3 | 45:55 Mixture of | 158 mM MD3 | 1245 | 1274 |
| | MD3 and 597 mM | 328 mM Phosphate | | |
| | Potassium Phosphate | (2.1 eq) | | |
| | Buffer, pH 7.0 (7- | | | |
| | day) | | | |
| 4 | 50:50 Mixture of | 177 mM MD3 | 1004 | 1034 |
| | MD3 and 380 mM | 190 mM Phosphate | | |
| | Potassium Phosphate | (1.1 eq) | | |
| | Buffer, pH 7.0 | | | |

* The MD3 sample used for Study Nos. 1 and 4 were obtained from the same production lot. The MD3 sample used for Study Nos. 2 and 3 were obtained from the same production lot.

### Non-Clinical Safety of MD3 Formulations and Vehicle Controls

As described in Table 3, several distinct formulations were tested in non-GLP (good laboratory practice) toxicity studies. A maximum tolerated dose study in rats (Study No. 2) demonstrated that a formulation containing up to 70 mg/mL MD3 and an osmolality of around 1200 mOsm/kg could be tolerated for a single dose without obvious clinical effects. However, similar formulations dosed daily for 7-days showed significant clinical and histopathological findings of toxicity. Closer examination of the data revealed that the MD3 formulations dosed for a short time were not nearly as toxic as the same formulations dosed for longer times. The toxicity observations could reasonably be associated with MD3; however, results from the vehicle control (potassium phosphate in water with NaCl added to match osmolality) produced similar toxicity results. In contrast, an isotonic saline group dosed for nearly 6 hours was well-tolerated. Taken together, contributions of potassium phosphate, osmolality, and dosing time on toxicity could not be deconvoluted.

Interestingly, a similar inhalation study in dogs using slightly lower osmolality formulations (850 mOsm/kg), was much better tolerated with few histopathological findings overall. The vehicle control was very well tolerated despite being dosed for 4 hours as was the isotonic saline control. While it is impossible to completely de-convolute formulation toxicity from MD3-induced toxicity from these two studies in different animal species, the results imply that minimizing the osmolality of the formulation and mitigating the time required for dosing is important to limiting overall toxicity during non-clinical inhalation studies.

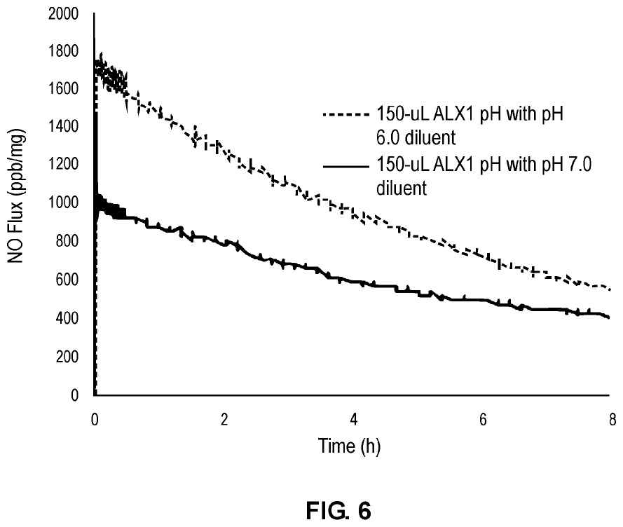

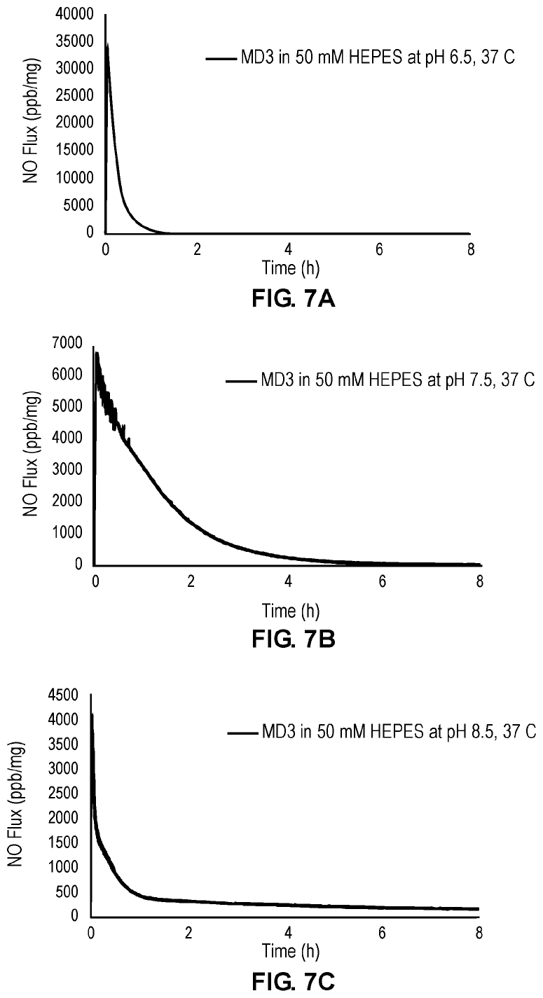

Based on the obtained data, the GLP-toxicity studies were designed to deliver the desired dose of MD3 with individual formulations optimized to minimize osmolality and dosing time. Because the phosphate concentration is dependent on the MD3 level, and the osmolality is dependent on the combination of MD3 and phosphate, MD3 formulation concentrations are selected to achieve the required dose with as close to an isotonic solution and in the minimal amount of exposure time as possible. FIG. 4 shows the impact of hypothetical compound (MD3) formulations on solution osmolality at a fixed concentration of buffering agent. FIG. 5 shows several different formulations (individual dots) selected to achieve the doses indicated in the top panel, as generated using computational studies. In FIG. 5, the generated values are linear permutations of each other and are based on the formulation strength and aerosol data generated and described herein.

In general, increased dosing times for each group would allow for lower MD3 formulation concentrations, and thus lower phosphate and osmolality. As demonstrated, the formulation stability can be optimized to the required dosing time by adjusting the phosphate concentration (top dot in each set—4 hour formulation stability; middle dot in each set—6 hour formulation stability; and bottom dot in each hours—8 hour formulation stability). At a range of doses, including at the doses indicated in FIG. 5, isotonic formulations with reasonable dosing times are achievable for MD3. While the doses indicated in FIG. 5 are tailored for animal dosing, the doses are modified for administration to humans and are significantly lower.

### Conclusion

The MD3 solution for nebulization has been optimized for simplicity and efficiency of running nonclinical toxicology studies. The formulation pH, buffer components and concentrations, and tonicity have been optimized to deliver MD3 as safely and robustly as possible.

### Example 2: NO Delivery at Varying pH Levels in Phosphate Buffers

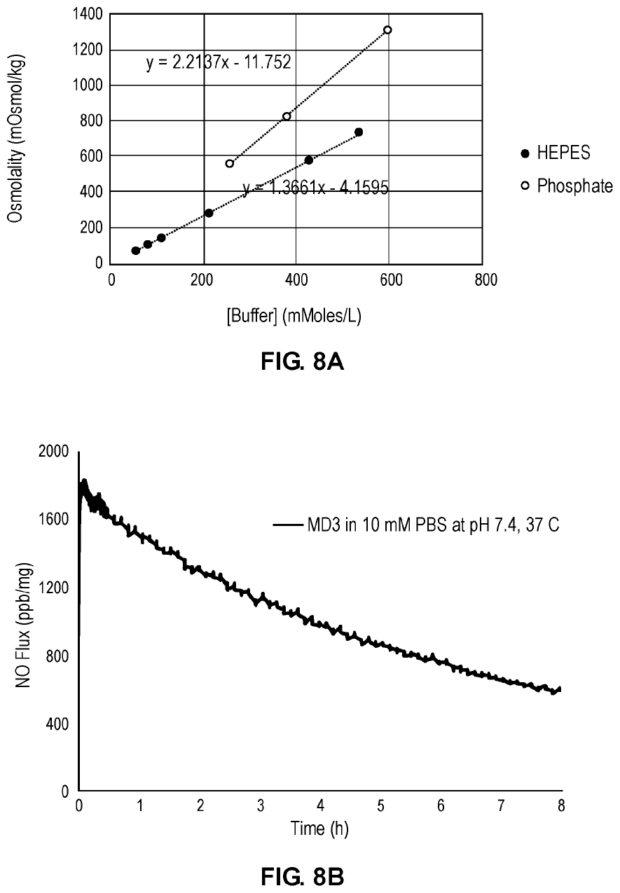

Two MD3 formulations were prepared as described above, each containing MD3 at a concentration of 28 mg/mL, with one formulation prepared at pH 6.0 and the other formulation prepared at pH 7.0 using 80 mM phosphate buffer. The formulations were prepared such that the delivered dose of MD3 was 11 mg and the deposited dose of MD3 was 4.4 mg. The deposited dose of MD3 corresponds to a local lung concentration of MD3 of 0.17 mg/mL. At various time points over a period of 8 hours, the NO flux was recorded for each solution to understand how the pH of the formulation impacts NO delivery. The results are shown in FIG. 6 and Table 4.

TABLE 4

| Delivered | | Max. | Max. | NO at 4 | NO at 4 | NO at 8 | NO at 8 |
|---|---|---|---|---|---|---|---|
| Dose | Lung | Flux | Flux | hours | hours | hours | hours |
| MD3 | Conc. | (ppb/mg) | (ppb/mg) | (μmol/mg) | (μmol/mg) | (μmol/mg) | (μmol/mg) |
| (mg) | (mg/mL) | pH 7 | pH 6 | pH 7 | pH 6 | pH 7 | pH 6 |
| 11 | 0.17 | ~1000 | ~1750 | 1.79 | 2.92 | 2.93 | 4.59 |

As shown in FIG. 6 and Table 4, NO delivery within 4 hours increased by up to 63% when formulating at pH 6.0 as compared to pH 7.0.

### Example 3: NO Delivery at Varying pH Levels in HEPES Buffers

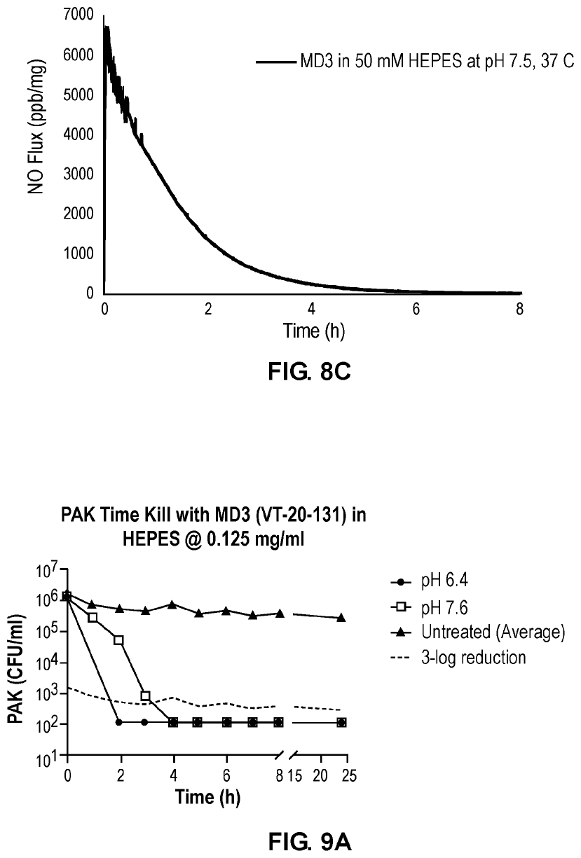

Three MD3 solutions were prepared, each containing between 0.25 and 0.50 mg of MD3 mixed with 30 mL of 50 mM HEPES buffer, at pH values of 6.5, 7.5, and 8.5. At various time points over a period of 8 hours, the NO flux (normalized on a per mg basis) was recorded for each solution at a temperature of 37° C. to understand how the pH of the solution impacts NO delivery. The results, which are shown in FIGS. 7A-C and Table 5, demonstrate that the NO flux increased with lower pH.

TABLE 5

| MD3 in 50 mM | | | |
|---|---|---|---|
| HEPES | [NO]ₘₐₓ | t_{1/2} | T[NO]_{4 h} (μmol/mg) |
| pH 6.5 | ~33800 | 0.22 | 5.34 |
| pH 7.5 | ~6500 | 1.01 | 4.65 |
| pH 8.5 | ~4100 | 8.80 | 1.22 |

### Example 4: Comparison of NO Release in HEPES and Phosphate Buffers

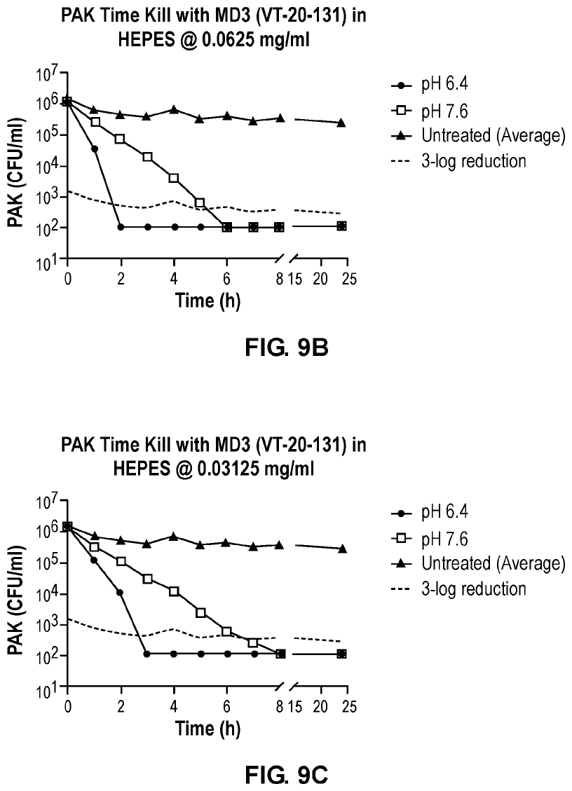

The effect of buffer concentration on osmolality was measured for HEPES and potassium phosphate buffer systems and plotted in FIG. 8A. With HEPES, a higher buffer concentration can be achieved with a lower osmolality (slope 1.37 mOsmol/Kg per mMoles/L HEPES), as compared to phosphate buffers (slope 2.21 mOsmol/Kg per mMoles/L phosphate).

Two MD3 solutions were prepared, each containing between 0.25 and 0.50 mg of MD3 mixed with 30 mL of 50 mM HEPES buffer (pH 7.5) or 30 mL of 10 mM phosphate buffered saline (pH 7.4). The NO release profile (normalized on a per mg basis) was measured for each of these solutions at 37° C.

At various time points over a period of 8 hours, the NO flux was recorded for each solution to understand how the pH of the formulation impacts NO delivery. The results, which are shown in Table 6 and by comparing FIGS. 8B and 8C, demonstrate that the NO flux increased in the solution including HEPES buffer as compared to phosphate buffer.

FIGS. 8B and 8C contain graphs showing the impact of buffer (phosphate buffer, FIG. 8B; HEPES, FIG. 8C) on NO flux over an eight-hour period for a MD3 containing composition.

TABLE 6

| MD3 | | | T[NO]_{1 h} | T[NO]_{4 h} | T[NO]_{8 h} |
|---|---|---|---|---|---|
| Concentration | [NO]ₘₐₓ | t_{1/2} | (μmol/mg) | (μmol/mg) | (μmol/mg) |
| 10 mM PBS | ~1800 | 5.42 | 1.00 | 3.09 | 4.86 |
| 50 mM | ~6500 | 1.01 | 2.72 | 4.65 | 4.87 |
| HEPES | | | | | |

### Example 5: PAK Time Kill with Compound (MD3) in HEPES and Phosphate Buffers

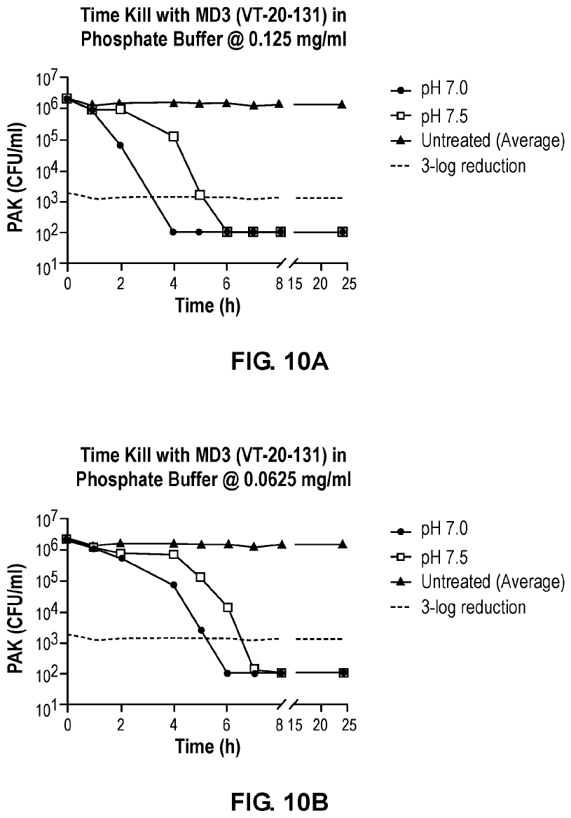

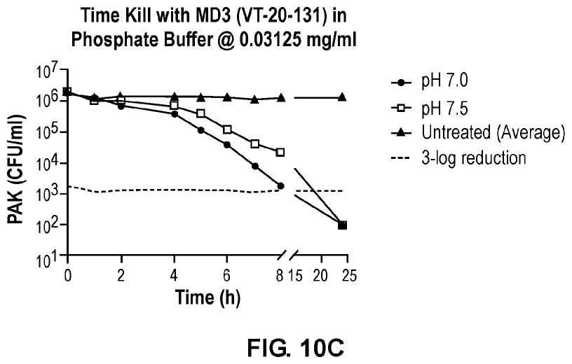

Pseudomonas aeruginosa strain K (PAK) time to kill experiments were performed using compositions of compound (MD3) in HEPES buffer and compound (MD3) in phosphate buffers, at varying concentrations of 0.125 mg/mL, 0.0625 mg/mL, and 0.03125 mg/mL for each buffer. Each study was performed using the composition at two pH values (pH 6.4 and 7.6 for HEPES, and pH 7.0 and 7.5 for phosphate), along with untreated PAK. The results of the experiments are shown in FIGS. 9A-C and 10A-C. FIGS. 9A-C contain graphs showing the time to kill Pseudomonas aeruginosa strain K (PAK) using varying concentrations of compound (MD3) in HEPES buffer, including 0.125 mg/mL (FIG. 9A), 0.0625 mg/mL (FIG. 9B), and 0.03125 mg/mL (FIG. 9C). FIGS. 10A-C contain graphs showing the time to kill Pseudomonas aeruginosa strain K (PAK) using varying concentrations of compound (MD3) in phosphate buffer, including 0.125 mg/mL (FIG. 10A), 0.0625 mg/mL (FIG. 10B), and 0.03125 mg/mL (FIG. 10C). As demonstrated by comparing the time kill data between the HEPES and phosphate buffers at specific concentrations (i.e., FIG. 9A compared to FIG. 10A, FIG. 9B compared to FIG. 10B, and FIG. 9C compared to FIG. 10C), the buffer used in the formulation impacts the efficacy of the compound. The compound (MD3) killed PAK faster in HEPES buffer compared to phosphate buffer at a pH of 7.5.

### Example 6: PAK Time Kill with Compound (MD3) in HEPES Buffers at Varying Concentrations and pH Values

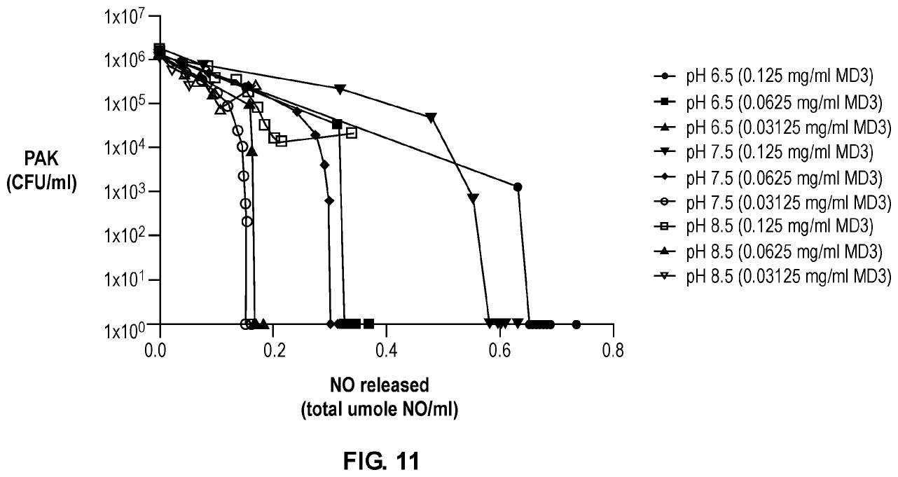

Pseudomonas aeruginosa strain K (PAK) time to kill experiments were performed using compositions of compound (MD3) in HEPES buffer, at varying concentrations of 0.125 mg/mL, 0.0625 mg/mL, and 0.03125 mg/mL. Each concentration was tested at a pH of 6.5, 7.5, and 8.5 at a temperature of 37° C. The nitric oxide (NO) release needed to kill PAK was measured and is shown in FIG. 11. As shown in FIG. 11, bacterial killing is concentration- and pH-dependent. However, bacterial killing is not dependent on total NO released. FIG. 10 shows, for example, that up to ˜0.4 μmol NO/mL is released by 0.125 mg/mL of compound (MD3) at pH 8.5 without killing PAK; however, half that amount kills PAK at a pH of 6.5 and 7.5. Not to be bound by theory, antimicrobial efficacy can be impacted by NO flux.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A composition, comprising:
a phosphate buffering agent; and
an aqueous carrier,
wherein the pH of the composition is maintained in a range of from 5.5 to 8.5 and the osmolality of the composition is from 270 mOsm/kg to 1300 mOsm/kg.

2. The composition of claim 1, wherein the phosphate buffering agent comprises potassium phosphate.

3. The composition of claim 1, further comprising an active pharmaceutical ingredient.

4. The composition of claim 3, wherein the active pharmaceutical ingredient is a water-soluble active pharmaceutical ingredient.

5. The composition of claim 3, wherein the active pharmaceutical ingredient comprises a mucolytic agent, an antibiotic, an antiviral, a corticosteroid, a monoclonal antibody (mAb), or an antifungal.

6. The composition of claim 3, wherein the active pharmaceutical ingredient comprises a nitric oxide (NO) releasing compound.

7. The composition of claim 6, wherein the nitric oxide (NO) releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation.

8. The composition of claim 7, wherein the compound has the following structure: 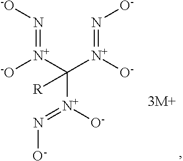
wherein
R is hydrogen, deuterium, C₁₋₁₂ alkyl, aryl, heteroaryl, alkylaryl, arylalkyl, or carbonyl, optionally substituted with one or more substituents, wherein the substituents are independently selected from the group consisting of —OH, —NH₂, —OCH₃, —C(O)OH, —CH₂OH, —CH₂OCH₃, —CH₂OCH₂CH₂OH, —OCH₂C(O)OH, —CH₂OCH₂C(O)OH, —CH₂C(O)OH, —NHC(O)—CH₃, —C(O)O((CH₂)ₐO)_{b}—H, —C(O)O((CH₂)ₐO)_{b}—(CH₂)_{c}H, —C(O)O(C₁₋₅alkyl), —C(O)—NH—((CH₂)_{d}NH)ₑ—H, —C(O)—NH—((CH₂)_{d}NH)ₑ—(CH₂)_{f}H, —O—((CH₂)ₐO)_{b}—H, —O—((CH₂)ₐO)_{b}—(CH₂)_{c}H, —O—(C₁₋₅alkyl), —NH—((CH₂)_{d}NH)ₑ—H, and —NH—((CH₂)_{d}NH)ₑ—(CH₂)_{f}H;
a, b, c, d, e, and f are each independently selected from an integer of 0, 1, 2, 3, 4, 5, 6, 7, 8, 9, or 10; and
M+ is a pharmaceutically-acceptable cation, wherein a ratio of the compound to the cation is such that the overall net charge of the compound is neutral.

9. The composition of claim 8, wherein the cation is selected from the group consisting of sodium, potassium, lithium, calcium, magnesium, ammonium, and substituted ammonium.

10. The composition of claim 8, wherein the compound has the following structure:

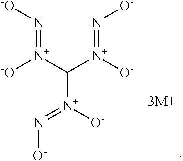

11. The composition of claim 8, wherein the compound has the following structure:

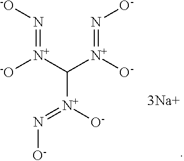

12. The composition of claim 8, wherein a molar equivalents concentration ratio of the phosphate buffering agent to the compound in the composition is at least 0.1:1.

13. The composition of claim 1, wherein the phosphate buffering agent maintains the pH of the composition in a range of from 5.5 to 8.0.

14. (canceled)

15. (canceled)

16. (canceled)

17. The composition of claim 1, wherein the composition has an osmolality of 270 mOsm/kg to 1300 mOsm/kg.

18. (canceled)

19. (canceled)

20. (canceled)

21. The composition of claim 1, wherein the phosphate buffering agent is substantially free from one or more of sodium phosphate, carbonate buffers, hydrochloric acid, sulphuric acid, or citric acid.

22. (canceled)

23. (canceled)

24. The composition of claim 1, further comprising one or more additives.

25. The composition of claim 24, wherein the one or more additives comprises one or more preservatives, salts, chelators, viscosity modifiers, stabilizers, surfactants, antioxidants, or cosolvents.

26. The composition of claim 1, wherein the molar equivalents concentration ratio of the phosphate buffering agent to the compound in the composition is at least 0.4:1.

27. (canceled)

28. (canceled)

29. (canceled)

30. The composition of claim 1, wherein the compound is present in an amount of from 0.1 mg/mL to 200 mg/mL.

31. (canceled)

32. The composition of claim 1, wherein the compound has a total releasable NO storage in a range of 0.1-23.0 μmol of NO per mg of the compound.

33. The composition of claim 1, wherein the compound has a NO release half-life in the range of 0.01-24 hours.

34. The composition of claim 1, wherein the compound has a total duration of NO release in a range of 0.1-60 hours.

35. The composition of claim 1, wherein the compound has a total NO release of 0.1-8.0 μmol of NO per mg of the compound after 4 hours of release initiation.

36. The composition of claim 1, wherein the composition is an inhalable composition.

37. A stable composition of a diazeniumdiolate compound in aqueous conditions, comprising:
a composition comprising a phosphate buffering agent, an aqueous carrier, and a nitric oxide releasing compound comprises at least two diazeniumdiolate groups on one carbon atom, each having a charge and each with an associated pharmaceutically-acceptable cation to balance the charge on the diazeniumdiolate groups, which compound has a molecular weight below 500 g/mol, not including the associated pharmaceutically-acceptable cation, wherein the pH of the composition is maintained in a range of from 5.5 to 8.5 and the osmolality of the composition is from 270 mOsm/kg to 1300 mOsm/kg.

38. A method for treating a respiratory disease in a subject, comprising administering to the subject an effective amount of a composition of claim 1.

39. The method of claim 38, wherein the composition is administered by inhalation, orally, or intravenously.

40. The method of claim 38, wherein the composition is administered using a nebulizer, a metered dose inhaler, or a dry powder inhaler.

41. (canceled)

42. (canceled)

43. The method of claim 38, wherein the respiratory disease comprises a chronic lung infection or an acute lung infection.

44. The method of claim 38, wherein the subject has asthma, chronic obstructive pulmonary disease, emphysema, acute bronchitis, cystic fibrosis, pneumonia, bronchiectasis, or bronchiolitis.
